(19) United States
(12) Patent Application Publication
(10) Pub. No.: US 2025/0195093 A1
(43) Pub. Date: Jun. 19, 2025

(54) METHODS, SYSTEMS, AND DEVICES FOR CENTERING AN ASPIRATION CATHETER WITHIN A BODY LUMEN

(71) Applicant: Walk Vascular, LLC, Irvine, CA (US)

(72) Inventors: Randolf von Oepen, Aptos, CA (US); Matthew J. Gillick, Murrieta, CA (US); Gioconda Alvarado, San Jose, CA (US)

(21) Appl. No.: 18/978,310

(22) Filed: Dec. 12, 2024

Related U.S. Application Data
(60) Provisional application No. 63/610,219, filed on Dec. 14, 2023.

(51) Int. Cl. A61B 17/22; A61B 17/00; A61B 17/3203

(52) U.S. Cl. CPC A61B 17/22; A61B 17/00234; A61B 17/32037; A61B 2017/00238; A61B 2017/00367; A61B 2017/00544; A61B 2017/00557; A61B 2017/00778; A61B 2017/00867; A61B 2017/22068; A61B 2017/22079; A61B 2217/005

(57) ABSTRACT

Disclosed are embodiments of an aspiration catheter comprising a centering assembly for preventing vessel tissue from being drawn into the aspiration lumen and/or into close proximity to an injection of pressurized fluid from a supply lumen during aspiration of a thrombus. The centering assembly may include one or more balloons mounted adjacent to the distal end of the aspiration catheter and which can be selectively inflated to center the distal end of the aspiration catheter within vessel lumen and space the distal end apart from the vessel wall.

### CROSS-REFERENCE TO RELATED APPLICATIONS

This application claims priority to and the benefit of U.S. Provisional Patent Application Ser. No. 63/610,219, filed Dec. 14, 2023, and entitled “METHODS, SYSTEMS, AND DEVICES FOR CENTERING AN ASPIRATION CATHETER WITHIN A BODY LUMEN,” the disclosure of which is incorporated herein by this reference.

### BACKGROUND OF THE INVENTION

### Technical Field

The present disclosure pertains generally to medical devices and methods of their use. More particularly, the present invention pertains to aspiration and thrombectomy devices and methods of use thereof.

### Description of the Related Art

Several devices and systems already exist to aid in the removal of thrombotic material. These include simple aspiration tube type devices using vacuum syringes to extract thrombus into the syringe, simple flush-and-aspirate devices, more complex devices with rotating components that pull in, macerate and transport thrombotic material away from the distal tip using a mechanical auger, and systems that use very high pressure to macerate the thrombus and create a venturi effect to flush the macerated material away.

All of the devices described above have limitations as a result of individual design characteristics. For example, while performing clot aspiration, a thrombectomy catheter may draw tissue, such as a vessel wall, into the catheter in a way that can cause tissue damage.

As such, what is needed are systems, methods, and devices, that limit or prevent tissue from being drawn into a thrombectomy catheter.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Embodiments of the present invention provide systems, methods, and devices for aspirating thrombus or material within a patient's body.

Implementations of the present invention solve one or more problems in the art with systems, methods, and devices for aspirating thrombus or material within a patient's body. For instance, implementations of the present invention relate to aspiration systems that prevent vessel tissue from being drawn into an aspiration lumen of an aspiration catheter and/or coming into close proximity to an orifice for jetted fluid from a supply lumen during aspiration. In one implementation, the aspiration system includes an elongate shaft, a supply lumen, an aspiration lumen, an orifice, and a centering assembly. The elongate shaft is configured for placement within a blood vessel and has a distal end and an outer surface. The supply lumen and the aspiration lumen each extend along the shaft. The supply lumen has a proximal end and a distal end and the aspiration lumen has a proximal end and a distal opening. The orifice is at or near the distal end of the supply lumen and is configured to allow injection of pressurized fluid into the aspiration lumen at or near the distal end of the aspiration lumen when the pressurized fluid is delivered through the supply lumen. The centering assembly is configured for centering the distal end of the elongate shaft within the blood vessel to prevent the blood vessel from being drawn into the aspiration lumen and coming into close proximity to the orifice of the supply lumen during aspiration.

In another configuration, an aspiration system includes an elongate shaft, a supply lumen, an aspiration lumen, an orifice, and a centering assembly. The elongate shaft is configured for placement within a blood vessel and has a distal end with an opening at the distal end. The supply lumen and the aspiration lumen each extend along the shaft. The supply lumen has a proximal end and a distal end and the aspiration lumen has a proximal end and a distal opening. The orifice is at or near the distal end of the supply lumen and is configured to allow injection of pressurized fluid into the aspiration lumen at or near the distal end of the aspiration lumen when the pressurized fluid is delivered through the supply lumen. The centering assembly is disposed at the distal end of the elongate shaft and comprises one or more balloons that are selectively inflatable to center the distal end of the elongate shaft within the blood vessel to prevent the blood vessel from being drawn into the aspiration lumen and into close proximity to the orifice of the supply lumen during aspiration.

In another configuration, an aspiration system includes an elongate shaft, a supply lumen, an aspiration lumen, an orifice, and a centering assembly. The elongate shaft is configured for placement within a blood vessel and has a distal end with an opening at the distal end. The supply lumen and the aspiration lumen each extend along the shaft. The supply lumen has a proximal end and a distal end and the aspiration lumen has a proximal end and a distal opening. The orifice is near the distal end of the supply lumen and is configured to allow injection of pressurized fluid into the aspiration lumen at or near the distal end of the aspiration lumen when the pressurized fluid is delivered through the supply lumen. The centering assembly is disposed at the distal end of the elongate shaft and includes a plurality of legs disposed adjacent to the distal end of the elongate shaft. The plurality of legs are movable between a retracted configuration and a deployed configuration to center the distal end of the elongate shaft within the blood vessel to prevent the blood vessel from being drawn into the aspiration lumen and into close proximity to the orifice of the supply lumen during aspiration.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a plan view of exemplary disposable components of a system for aspirating thrombus according to an implementation of the present disclosure.

FIG. 2 is a sectional view of an exemplary distal end of the aspiration catheter of the system for aspirating thrombus of FIG. 1 according to an implementation of the present disclosure.

FIG. 3 is a detail view of an exemplary y-connector of the aspiration catheter of the system for aspirating thrombus of FIG. 1 according to an implementation of the present disclosure.

FIG. 4 is a plan view of exemplary disposable components of a system for aspirating thrombus according to an implementation of the present disclosure.

FIG. 5 is a perspective view of an exemplary system for aspirating thrombus of FIG. 3 according to an implementation of the present disclosure.

FIG. 6 is a perspective view of an exemplary aspiration catheter according to an implementation of the present disclosure.

FIG. 7 is a sectional view of an exemplary aspiration catheter according to an implementation of the present disclosure.

FIG. 8 illustrates the exemplary aspiration catheter within a body lumen aspirating thrombus according to an implementation of the present disclosure.

FIGS. 9A-9B illustrate an exemplary aspiration catheter with a centering assembly according to an implementation of the present disclosure.

FIG. 10 illustrates an exemplary aspiration catheter with a centering assembly according to an implementation of the present disclosure.

FIGS. 11A-11B illustrate an exemplary aspiration catheter with a centering assembly according to an implementation of the present disclosure.

FIGS. 12A-12B illustrate an exemplary aspiration catheter with a centering assembly according to an implementation of the present disclosure.

FIG. 13 illustrates a control and balloon inflation system assembly according to an implementation of the present disclosure.

FIGS. 14A-14B illustrate an exemplary aspiration catheter with a centering assembly according to an implementation of the present disclosure.

FIGS. 15A-15B illustrate an exemplary aspiration catheter with a centering assembly according to an implementation of the present disclosure.

FIGS. 16A-16B illustrate an exemplary aspiration catheter with a centering assembly according to an implementation of the present disclosure.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to the preventing tissue, such as tissue of a lumen (e.g., a vessel wall) receiving an aspiration catheter, from being drawn into an aspiration lumen of the aspiration catheter and coming into close proximity to an orifice for jetted fluid from a supply lumen during aspiration. A catheter centering assembly or a means for centering can position the distal opening of a catheter near the center of a body lumen and away from a body wall, which can limit or prevent the body wall from being brought drawn into the aspiration catheter or sprayed with the fluid jetted or ejected from the supply lumen, thereby preventing tissue damage during aspiration of thrombus. Thus, the aspiration process is safer for a patient.

While the present disclosure will describe particular implementations for preventing tissue from being drawn into an aspiration lumen of the aspiration catheter and coming into close proximity to an orifice for jetted fluid from a supply lumen during aspiration, it should be understood that the devices, systems, and method described herein may be applicable to other uses. Additionally, elements described in relation to any embodiment depicted and/or described herein may be combinable with elements described in relation to any other embodiment depicted and/or described herein.

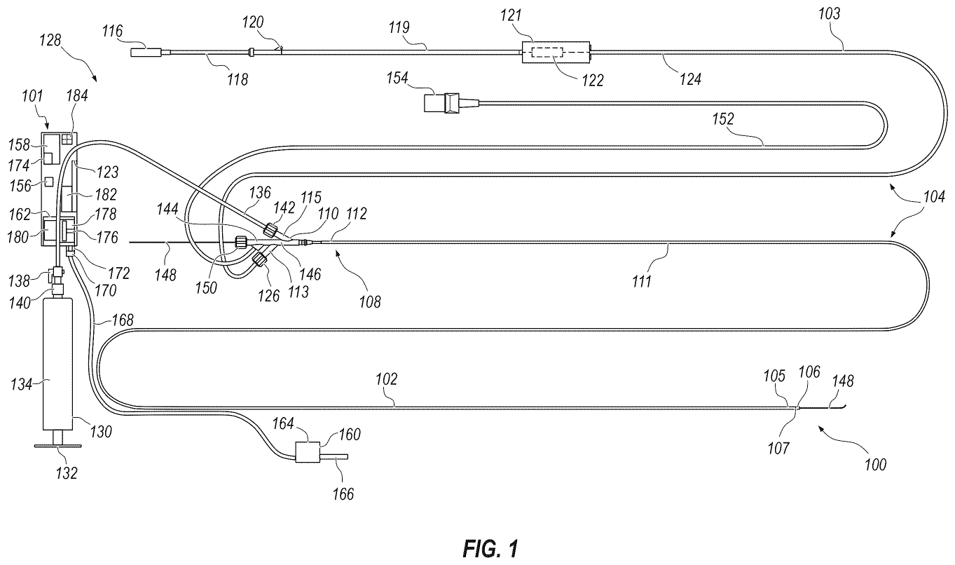

A system 100 for aspirating thrombus is illustrated in FIGS. 1-5. The system 100 for aspirating thrombus includes a pump 101, an aspiration catheter 102, and a tubing set 103. The aspiration catheter 102 and the tubing set 103 represent disposable components, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

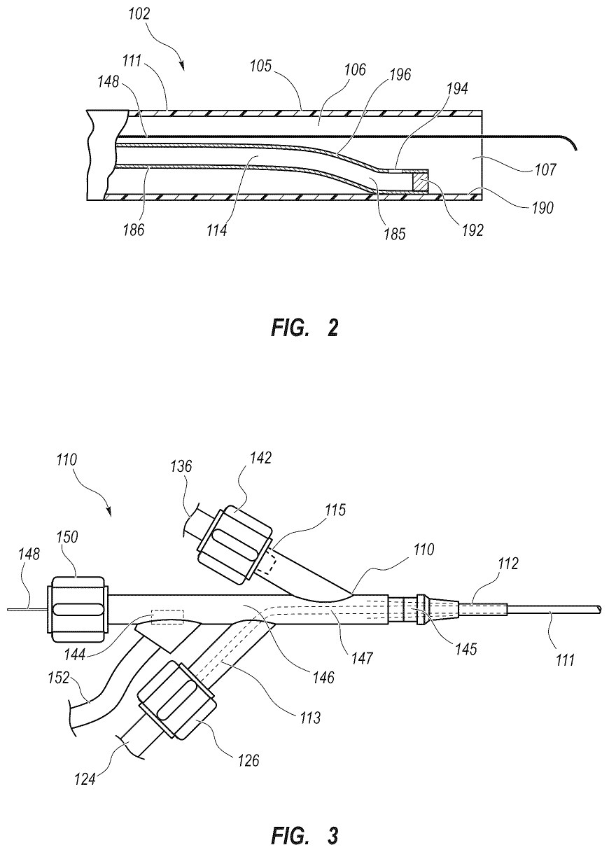

The aspiration catheter 102 has a distal end 105 and includes an over-the-wire guidewire lumen/aspiration lumen 106 extending between an open distal end 107 and a proximal end 108 comprising a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 includes a first female luer 113 which communicates with a catheter supply lumen 114 (FIG. 2), and a second female luer 115 which communicates with the guidewire lumen/aspiration lumen 106.

A spike 116 for coupling to a fluid source (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

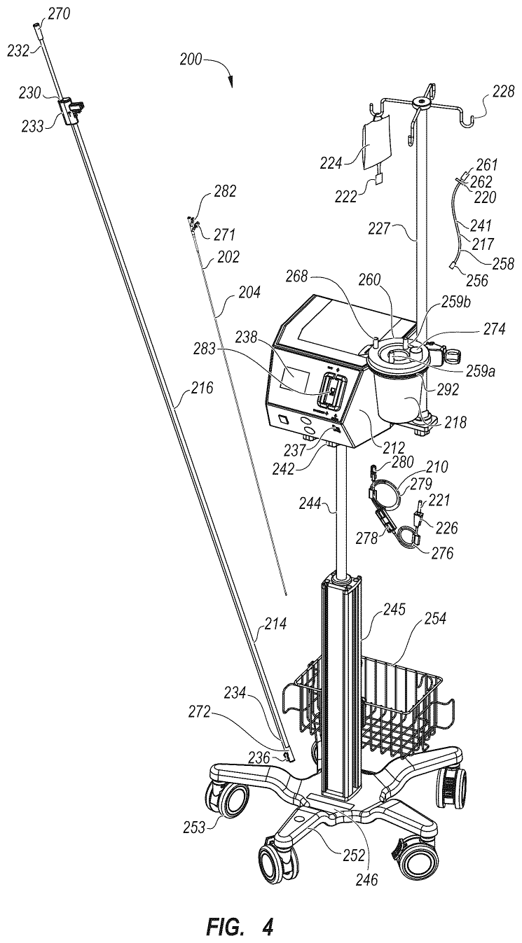

Accessories 128 are illustrated that are intended for applying a vacuum source, such as a syringe 130 having a plunger 132 and a barrel 134, to the aspiration lumen 106 of the aspiration catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 3, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector 154. The connector 154 is plugged into a socket 156 of the pump 101. Pressure related signals may be processed by a circuit board 158 of the pump 101. The pressure sensor or transducer 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump 101 may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure sensor or transducer 144 thus senses a negative pressure and sends a signal, causing the controller to start the motor 182 of the pump 101. As the effect via the electronics is substantially immediate, the motor 182 starts pumping almost immediately after the pedal 166 is depressed. As the pedal 166 of the foot pedal 160 is released, the controller 174 then causes the pinch valve 162 to close. The pressure sensor or transducer 144 thus senses that no negative pressure is present and the controller 174 causes the motor 182 of the pump 101 to shut off. Again, the effect via the electronics is substantially immediate, and thus the motor 182 stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the aspiration catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage both in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 2, a supply tube 186, which contains the catheter supply lumen 114, freely and coaxially extends within the over-the-wire guidewire lumen/aspiration lumen 106. At least a distal end 185 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 185 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torqueability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

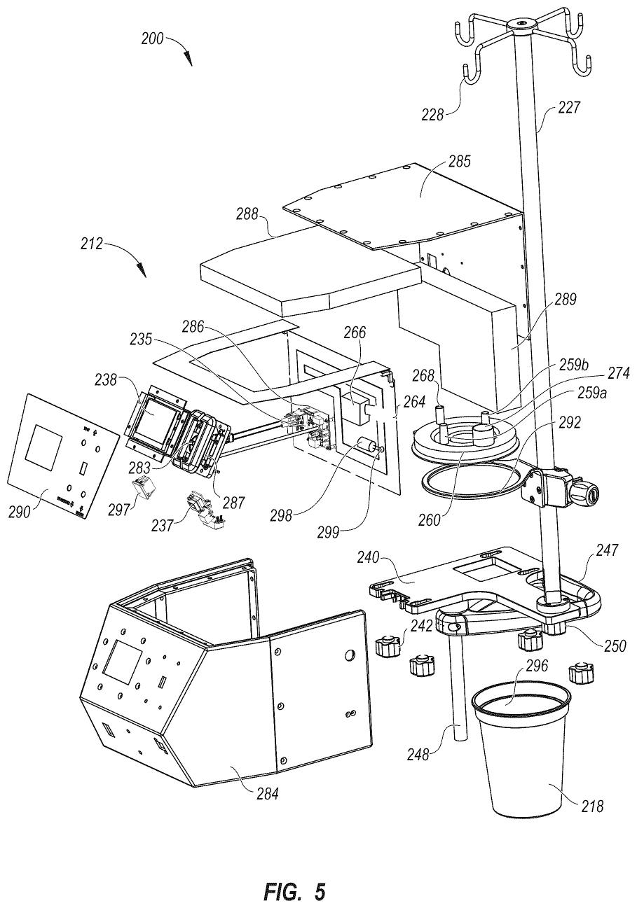

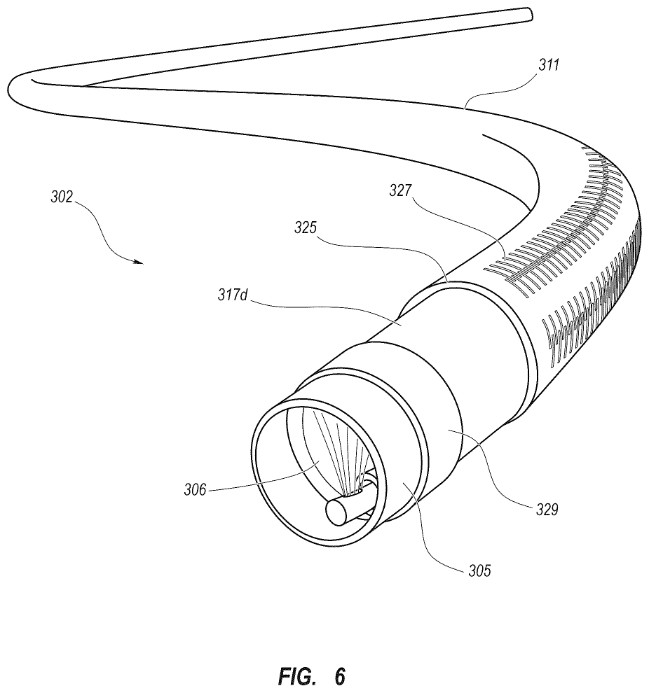

A system 200 for aspirating thrombus is illustrated in FIGS. 4-5. The system 200 for aspirating thrombus is similar to the system 100 and so the disclosure related to the system 100 is also applicable to the description of system 200. An aspiration catheter 202 is similar to the aspiration catheter 102 of FIGS. 1-3 and as such the description related to the aspiration catheter 102 of FIGS. 1-3 is also applicable to the description of the aspiration catheter 202.

The aspiration catheter 202 is configured for aspirating thrombus from peripheral vessels, but may also be configured with a size for treating coronary, cerebral, pulmonary or other arteries, or veins. The aspiration catheter 202/system 200 may be used in interventional procedures, but may also be used in surgical procedures. The aspiration catheter 202/system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIG. 3) and a guidewire/aspiration lumen 106, each extending along the shaft, the supply lumen 114 having a proximal end 147 and a distal end 185, and the aspiration lumen 106 having a proximal end 145 (FIG. 3) and an open distal end 107 (FIG. 2); and an orifice or opening 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114. In some embodiments, the orifice or opening 194 may be located proximal to the distal end 185 of the supply lumen 114. In some embodiments, the distal end 185 of the supply lumen 114 may comprise a plug 192. A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 4 within a lumen at a junction between a first aspiration tube 232 and a control 233. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259 on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 274 in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 121 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 121 may couple to the saddle 283 by clipping, friction fitting, vacuum fitting, or other means.

FIG. 5 illustrates aspects pertaining to the vacuum canister 218, in which aspirant (e.g., thrombus, blood, saline) that is evacuated from the patient through the aspiration lumen 106 is collected. The canister 218 may be held in a canister mount 292 carried by the IV pole 227, or alternatively carried by any other part of the system 200. A lid 260 is configured to cover a portion of the canister 218, such as in a snapping manner, to close an interior 296 of the canister 218. Alternatively, the lid 260 may couple to the canister 218 by screwing, clipping, friction fitting, or other means.

The lid 260 may comprise two or more ports, including the first port 268 and second port 259 for providing negative pressure/vacuum to the aspiration lumen 106. For example, the lid 260 may comprise two ports, three ports, four ports, or more than four ports. Sterile suction tubing 216 may be connected to the lid 260 of the vacuum canister 218 at a first port 268 for transmitting a negative pressure to the sterile suction tubing 216 and to the aspiration lumen 106 of the aspiration catheter 102. Non-sterile suction tubing 217 may be connected to the lid 260 of the vacuum canister 218 at a second port 259 for providing a negative pressure to the vacuum canister 218. A negative pressure may be provided to the non-sterile suction tubing 217 (and to sterile suction tubing 216 and the aspiration lumen 106 connected therewith) by a vacuum source (e.g., a vacuum pump or syringe). The system 200 may also comprise means for sealing the two or more ports of the lid 260 when not in use, such as one or more port caps. A filter may be placed over an entry to the second port 259 so as to prevent aspirant from traveling along the non-sterile suction tubing 217 from the vacuum canister 218 to the vacuum source.

The vacuum canister 218 preferably has a sufficient volumetric capacity for receiving all aspirant collected during the surgical procedure. Receptacles having a volumetric capacity of approximately 100 cubic inches, or receptacles having a diameter of approximately 5.0 inches and a height of approximately 7.0 inches, have been found to provide sufficient volumetric capacity.

Returning to FIG. 5, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

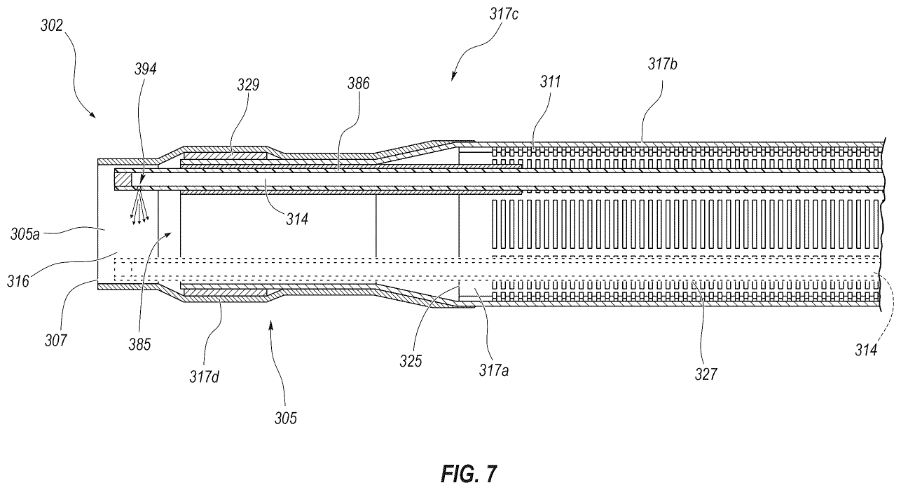

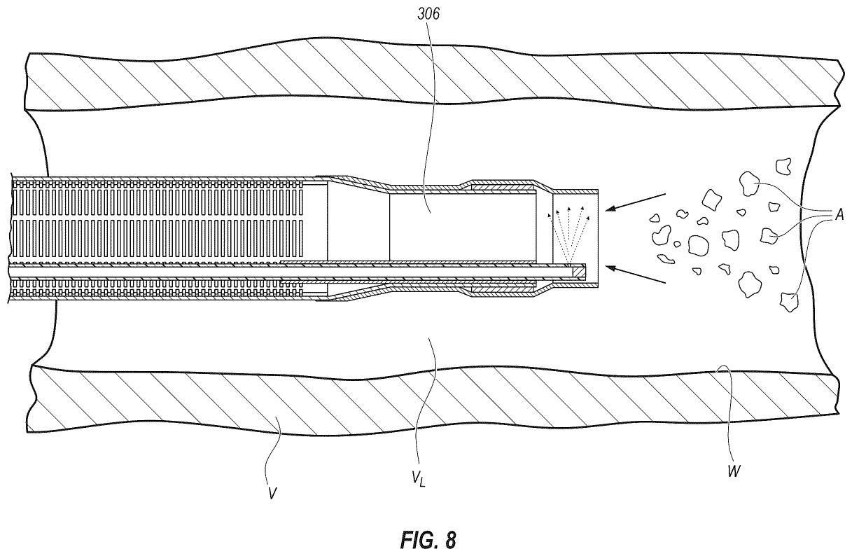

Turning to FIGS. 6-8 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 6-8.

As shown in more detail in FIGS. 6-8, aspiration catheter 302 includes an aspiration lumen 306 formed by a shaft 311, such as a hypotube, jacketed by a polymer jacket or a polymer jacket is laminated on the hypotube. For instance, a shaft body 317a is illustrated being jacket by a jacket 317b. A distal end 305 of the aspiration catheter 302 includes a multilayer structure. A portion 317c of the jacket 317b extends distally of a shaft distal end 325 of the shaft 311 to form part of the distal end 305. An outer layer or outer jacket 317d overlaps the jacket 317b, extends towards and overlaps the distal end 325 of the shaft 311, and forms the aspiration catheter distal end 305a or the distal most end of the aspiration catheter with the distal opening 307. The outer jacket 317d protects a distal portion 385 of a supply tube 386 containing a supply lumen 314. While reference is made of a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structures to form the shaft.

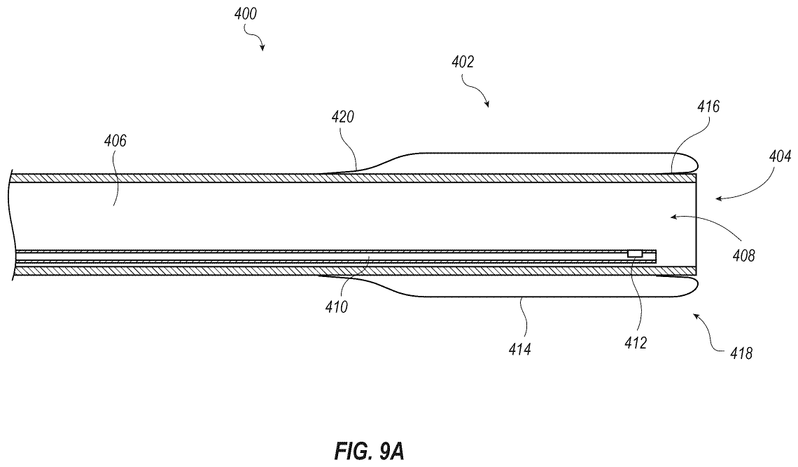

The supply lumen 314 may be configured to provide a high pressure fluid injection, such as saline, within the aspiration lumen 306 for macerating a thrombus as it is aspirated, such as illustrated in FIG. 8 where the aspiration catheter 302 is disposed within a vessel lumen VL of a vessel V and aspirant A is being drawing into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. The radiopaque ring 329 optionally encircles the aspiration catheter 302. In the illustrated configuration, the RO ring 329 is disposed between the jacket 317b and outer jacket 317d. The RO ring 329 can be formed of any suitable radiopaque material, such as tantalum, tungsten, platinum/iridium, gold, silver, and combinations or modifications thereof.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. Furthermore, the jacket 317b and outer jacket 317d can be formed of a variety of polymers and copolymers, plastics, PEBAX, HYTREL, rubber, Nylon, polyolefin, fluorinated polymers like FEP or PTFE, polyurethan, polyamides, and combinations or modifications thereof.

In some situations, during aspiration, damage to the vessel might occur because the vessel wall is drawn into the aspiration lumen 306 and comes into contact with the pressurized fluid injection (e.g., a high pressure saline spray at, for example, 650 psi) from supply lumen 314. A catheter centering assembly can prevent aspiration catheter 302 from damaging the vessel during aspiration. The catheter centering assembly can be configured to position the distal opening 307 of the aspiration lumen 306 near the center of the vessel lumen and spaced apart from the vessel wall, thereby limiting or preventing the vessel wall from being drawn into the aspiration lumen 306.

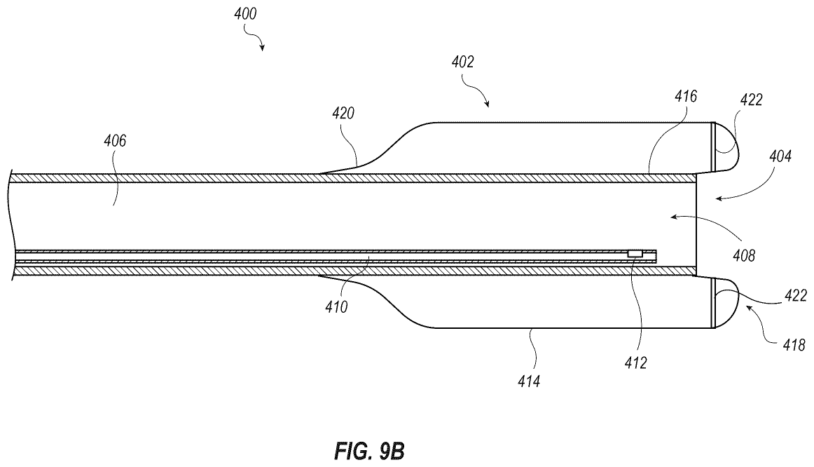

FIGS. 9A-12B relate to various centering assemblies, devices, features, means, and methods for limiting or preventing a vessel wall or other body tissue from being undesirably drawn into the aspiration lumen of an aspiration catheter. The centering assemblies, devices, means, and features may also be referred to as centering or spacing assemblies, devices, means, or features because they can be configured to center the distal end of the aspiration catheter within the vessel or other body lumen or space the distal end of the aspiration lumen catheter away from wall of the vessel or other body cavity.

FIGS. 9A and 9B illustrates an example implementation of an aspiration catheter 400 with a centering assembly 402 mounted adjacent to a distal end 404 of the aspiration catheter 400. The aspiration catheter 400 may be similar or identical to the other aspiration catheters disclosed herein. For instance, the aspiration catheter 400 may include a lumen 406 extending therethrough and an open end 408 at the distal end 404 thereof. Additionally, the aspiration catheter 400 may include a supply lumen 410 within the lumen 406 for delivering fluid through an opening or orifice 412.

In the illustrated implementation, the centering assembly 402 comprises a balloon 414 mounted on the outer surface of the aspiration catheter 400. The balloon 414 can extend around all or a substantial portion of the outer circumference of the distal end 404 of the aspiration catheter 400. The balloon 414 may be formed of any suitable material, including polyethylene (PE), polyurethane (PU), polyethylene terephthalate (PET), polyether block amide (PEBAX), nylon, silicon, and the like.

The balloon 414 may be selectively moved from an undeployed or deflated configuration to a deployed or inflated configuration. FIG. 9A illustrates the balloon 414 in the undeployed or deflated configuration and FIG. 9B illustrates the balloon 414 in the deployed or inflated configuration. In the undeployed or deflated configuration, the balloon 414 may conform to the shape of the outer surface of the aspiration catheter 400, such that the balloon 414 lays flat on the aspiration catheter 400. In other implementations, the balloon 414 may be folded or rolled on the outer surface of the aspiration catheter 400 when the balloon 414 is in the undeployed or deflated configuration.

In the illustrated embodiment, the distal end 416 of the balloon 414 is rolled or folded under and attached to the aspiration catheter 400. In the illustrated implementation, the distal end 416 is attached to the outer surface of the aspiration catheter 400. In other implementations, the distal end 416 of the balloon 414 may be rolled or folded into the open end 408 and attached to the interior surface of the aspiration catheter 400. In either case, rolling or folding the distal end 416 under can create a rounded distal region 418 of the balloon 414 when the balloon 414 is inflated as shown in FIG. 9B.

In some embodiments, the rounded distal region 418, when deployed or inflated, may be generally aligned with the terminal end of the aspiration catheter 400 or may be positioned proximal to the terminal end of the aspiration catheter 400. In the illustrated implementation, when deployed or inflated, the rounded distal region 418 extends distal to the terminal end of the aspiration catheter 400. Having the rounded distal region 418 extend distal to the terminal end of the aspiration catheter 400 may allow the rounded distal region 418 to act as a bumper to reduce the potential trauma from the terminal end of the aspiration catheter 400 engaging body tissue.

As shown in FIG. 9B, the rounded distal region 418 of the balloon 414 may include one or more valves 422. The valves 422 may extend through and open to opposing sides of the balloon 414. The valves 422 may be conduits passing through the balloon 414. As a result, fluid may pass through the valves 422. The valves 422 may limit or prevent the distal end of the catheter 400 from becoming suctioned against body tissue. For instance, during an aspiration procedure, fluid is drawn into the aspiration catheter 400 via a vacuum pressure as described herein. If the distal end of the catheter 400 is placed too close to body tissue, the vacuum pressure may lead to the distal end of the catheter 400 or the rounded distal region 418 of the balloon may form a seal with the body tissue, causing the catheter 400 to suction onto the body tissue. However, with the inclusion of the valves 422, the full vacuum pressure may not be applied to the body tissue because some of the vacuum pressure will be applied to the valves 422. Additionally, the fluid communication through the valves 422 can prevent the catheter 400 from becoming suctioned onto the body tissue or at least reduce the suction force, thereby limiting trauma to the body tissue.

In the illustrated embodiment, inner surface of the balloon 414 at the proximal end 420 thereof may be connected to the outer surface of the aspiration catheter 400. When the balloon 414 is in the deployed or inflated configuration, as shown in FIG. 9, the proximal end 420 may taper in the proximal direction, creating a ramp. The distal and proximal ends 416, 420 of the balloon 414 may be attached to the aspiration catheter 400 through any suitable modality, including heat bonding, laser or ultrasonic welding, adhesive, or the like. In some implementations, such as when the balloon 414 is formed of silicon, a suture or other string may be secured around the distal and/or proximal ends 416, 420 of the balloon 414 to help create a fluid tight seal between the balloon 414 and the aspiration catheter 400. Silicon can be difficult to bond to other materials in a way that creates a fluid tight seal, so using a suture or other string in addition to or as an alternative to heat bonding, laser or ultrasonic welding, or adhesives may create the desired fluid tight seal.

The interior of the balloon 414 may be in fluid communication with an inflation lumen. In some implementations, the inflation lumen can extend along the outer surface of the aspiration catheter 400 between an inflation device (discussed in more detail below) and the balloon 414. The inflation lumen may extend between the outer surface of the aspiration catheter 400 and the proximal end 420 of the balloon 414 to allow air, CO2, or other fluids to be delivered into or evacuated from the balloon 414 to inflate or deflate the balloon 414.

The balloon 414 may have various coatings applied thereto. Such coatings may include lubricants to facilitate movement of the balloon through the vessel lumen. When the balloon 414 is formed of a soft material, such as PU, Pebax, or silicon, it can be particularly useful to coat the balloon 414 with a lubricant, whether hydrophilic or hydrophobic, to enable the balloon 414 to move more easily through a body lumen. Such soft balloon materials can be sticky when not coated with a lubricant. Other coatings may improve the biocompatibility of the balloon 414, provide drug delivery, or have antimicrobial properties.

In use, the aspiration catheter 400 may be inserted into a vessel (or other body) lumen and advanced until the distal end 404 thereof is positioned adjacent or against thrombus. The balloon 414 may then be inflated. Inflation of the balloon 414 will center the distal end 404 of the aspiration catheter 400 within the vessel lumen and space the open end 408 apart from the vessel wall. Thereafter, aspiration may be commenced. As noted herein, aspiration may include spraying fluid out of the orifice 412 to macerate the thrombus and evacuating (via vacuum) the macerated thrombus and sprayed fluid through the aspiration lumen 406. Because the open end 408 is spaced apart from the vessel wall, the vessel wall is less likely to be inadvertently drawn into the open end 408 of the aspiration catheter 400.

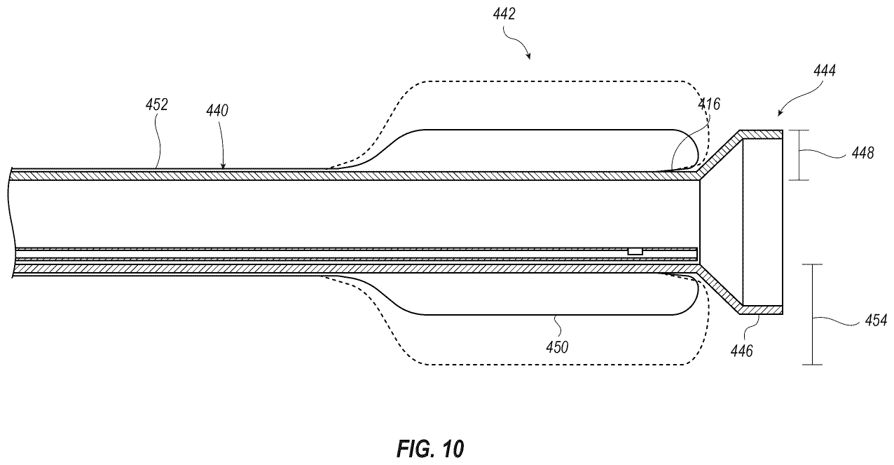

FIG. 10 illustrates another example implementation of an aspiration catheter 440 with a centering assembly 442 mounted adjacent to a distal end 444 of the aspiration catheter 440. The aspiration catheter 440 and the centering assembly 442 may be the same as or similar to the aspiration catheter 400 and centering assembly 402 in many respects. Accordingly, the following discussion of the implementation of FIG. 10 will focus on those features that are different from the implementation of FIG. 9.

The primary distinction between the implementations of FIGS. 9 and 10 is that the aspiration catheter 440 of FIG. 10 includes a distal end or tip 446 that is wider than the rest of the aspiration catheter 440. For instance, the tip 446 may have an outer diameter that is about 2 French, 4 French, 6 French, other dimension larger than the diameter of the rest of the aspiration catheter 440. This dimension 448 may be approximately equal to the outer dimension of the balloon 450 in the deflated state (the balloon 450 is shown in solid lines in the deflated state). Accordingly, the tip 446 and the balloon 450 may have approximately the same outer dimensions when the balloon 450 is deflated and while the aspiration catheter 440 and centering assembly 442 are being positioned within a vessel lumen.

The tip 446 may be formed of various materials, including relatively soft and/or pliable materials. In some implementations, the tip 446 is formed of the same material as the rest of the aspiration catheter 440. In other implementations, the tip 446 is formed of a material that is different from the rest of the catheter 440. Likewise, the tip 446 may be integrally formed with the rest of the aspiration catheter 440 or may be formed separately and attached thereto.

The implementation of FIG. 10 may also include a coaxial shaft 452 disposed about the aspiration catheter 440. The coaxial shaft 452 may include an inflation lumen that is in fluid communication with the balloon 450 to inflate or deflate the balloon 450. In the illustrated implementation, the coaxial shaft 452 may have an outer diameter that is approximately the same as that of the tip 446 and the uninflated balloon 450, which may facilitate positioning of thereon in a vessel lumen.

As shown in dashed lines in FIG. 10, the balloon 450 may extend from the outer surface of the aspiration catheter 440 a dimension 454. The dimension 454 may be large enough that the balloon 450 extends radially out beyond the tip 446 and the coaxial shaft 452 so that the inflated balloon 450 can engage the vessel wall and center the aspiration catheter 440 within the vessel lumen.

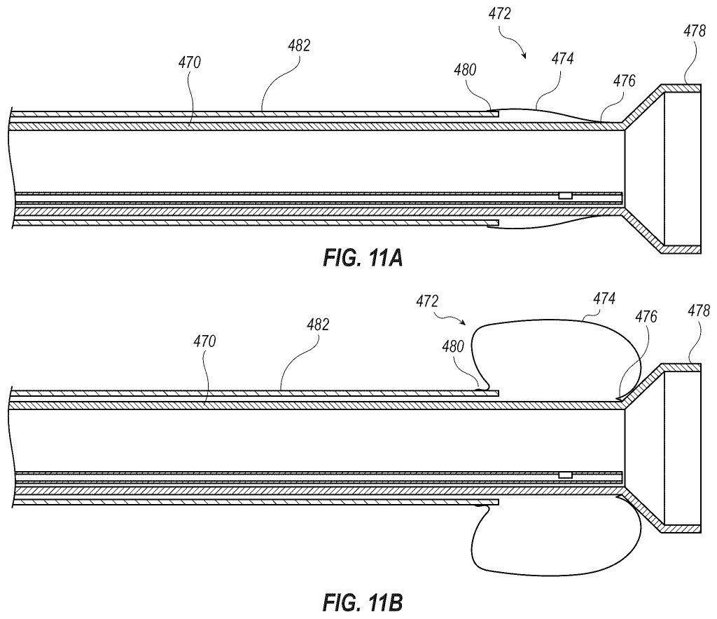

FIGS. 11A and 11B illustrate another example implementation of an aspiration catheter 470 with a centering assembly 472 mounted adjacent to a distal end 474 of the aspiration catheter 470. The aspiration catheter 470 and the centering assembly 472 may be the same as or similar to the aspiration catheter 440 and the centering assembly 442 in many respects. Accordingly, the following discussion of the implementation of FIGS. 11A and 11B will focus on those features that are different from the previous implementations.

The primary distinction between the implementation of FIG. 10 and the implementation of FIGS. 11A and 11B relates to the centering assembly 472. As can be seen in FIGS. 11A and 11B, the centering assembly 472 includes an inflatable sleeve or tube 474 (which may also be referred to as a balloon). The tube 474 includes a distal end 476 that is attached to the outer surface of the aspiration catheter 470. In the illustrated implementation, the distal end 474 is attached to the aspiration catheter 470 adjacent to or proximal of the wider tip 478, but the distal end 474 may be attached to the tip 478. The tube 474 includes a proximal end 480 that is attached to the outer surface of the coaxial shaft 482. Because the coaxial shaft 482 has a larger diameter than the aspiration catheter 470 (other than the wider tip 478), the distal end 474 and the proximal end 480 of the tube 474 are attached at different radial locations.

The coaxial shaft 482 may include an inflation lumen that is in fluid communication with the interior of the tube 474. Air or other fluids may be introduced into the interior of the tube 474 so as to inflate the tube 474 from the uninflated configuration of FIG. 11A to the inflated configuration of FIG. 11B. Similarly, the air or other fluid may be evacuated from the interior of the tube 474 to as to deflate the tube 474.

Similar to the implementation of FIG. 10, the tip 478 and the coaxial shaft 482 may have similar or the same outer dimensions. A portion of the uninflated tube 474 may have an outer dimension that is similar to the outer dimensions of the tip 478 and the coaxial shaft 482. In other implementations, at least a portion of the uninflated tube 474 may have an outer dimension that is smaller than that of the tip 478 and the coaxial shaft 482.

While the implementation of FIGS. 11A and 11B illustrate the aspiration catheter 470 as having a wider tip 478, there is merely exemplary. In other embodiments, the distal end of the aspiration catheter 470 may have a diameter that is the same as the rest of the catheter 470, similar to the aspiration catheter 400 of FIG. 9.

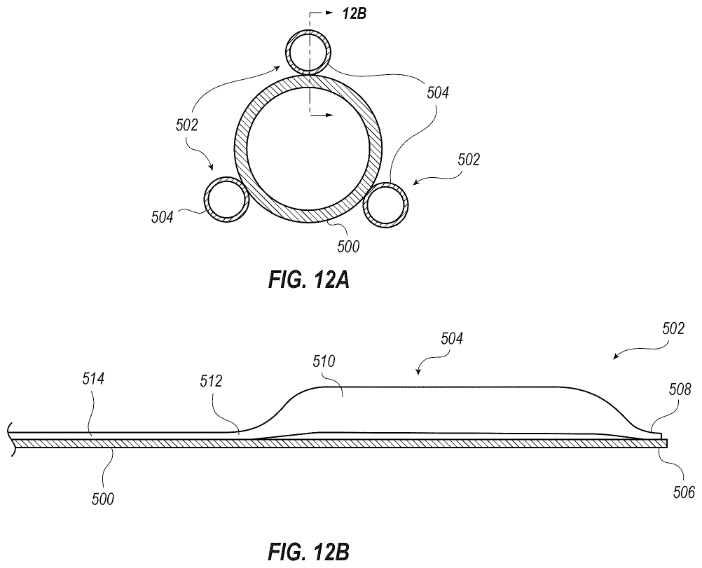

FIGS. 12A and 12B illustrate another example implementation of an aspiration catheter 500 with a centering assembly 502 mounted adjacent to a distal end 504 of the aspiration catheter 500. The aspiration catheter 500 and the centering assembly 502 may be the same as or similar to the other aspiration catheters and/or centering assemblies disclosed herein in many respects. Accordingly, the following discussion of the implementation of FIGS. 12A and 12B will focus on those features that are different from the previous implementations.

FIG. 12A illustrates an end cross sectional view of the aspiration catheter 500 and the centering assembly 502 and FIG. 12B illustrates a partial side cross sectional view thereof. In contrast to the previous implementations in which the centering assemblies extended around all or substantially off of the outer circumference of the aspiration catheter, the centering assembly 502 includes a plurality of balloons 504 disposed at discrete locations around the outer circumference of the aspiration catheter 500. The illustrated implementation includes three balloons 504 equally spaced around the outer circumference of the aspiration catheter 500. It will be appreciated that fewer or more than three balloons 504 may be used in other implementations.

In the illustrated implementation, each of the balloons 504 extends along a portion of the length of the aspiration catheter 500 near the distal end 506 thereof. As can be seen in FIG. 12B, each balloon 504 includes a distal section 508, an inflatable portion 510, and a proximal section 512. The distal and proximal sections 508, 512 may be attached to the outer surface of the aspiration catheter 500. In some cases, the inflatable portion 510 may also be attached to the aspiration catheter 500. The distal and proximal sections 508, 512 may be tapered or have smaller dimensions than the inflatable portion 510.

The proximal section 512 may be in fluid communication with an inflation lumen 514. The inflation lumen may be attached to the outer surface of the aspiration catheter 500 or may be a lumen extending through the aspiration catheter 500. In any case, the inflation lumen 514 may be configured to deliver or evacuate air or fluid to or from the balloon 504. When the balloons 504 are inflated, they may interact with the vessel wall so as to center the aspiration catheter 500 within the vessel lumen.

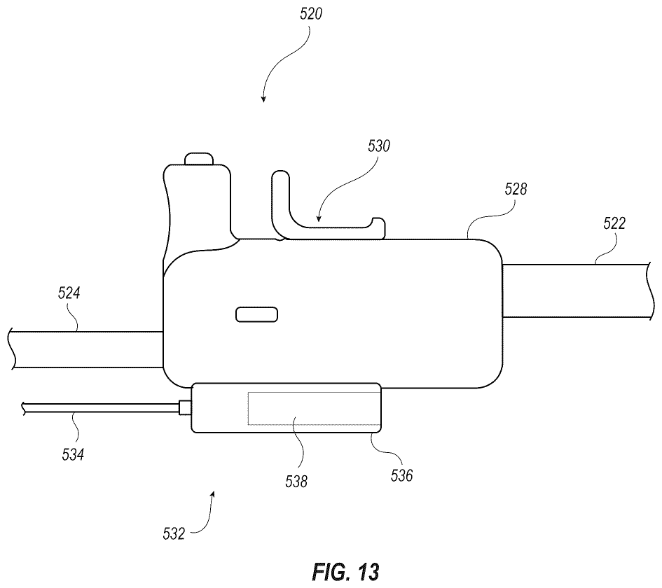

Attention is now directed to FIG. 13, which illustrates a control 520. The control 520 may include sections of suction tubing 522, 524 and a valve 526 (internal to the housing 528. The suction tubing 522 may be connected between the valve 526 and the SDU 212 and the suction tubing 524 may be connected between the valve 526 and any of the aspiration catheters disclosed herein that has a balloon attached thereto. The control 520 may include an input device 530 that allows a user to selectively open or close the valve 526 to start or stop aspiration through the aspiration catheter. Additional details regarding example embodiments of the control 520 may be found in U.S. Pat. Publ. No. 2022/0257268, published Aug. 18, 2022, and entitled, “Systems and Methods for Removal of Blood and Thrombotic Material,” which is incorporated herein by reference in its entirety.

As shown in FIG. 13, the control 520 may also include or be associated with a balloon inflation system 532. The balloon inflation system 532 may be in fluid communication with any of the balloons disclosed herein to inflate or deflate the balloons. For instance, the balloon inflation system 532 may be connected to the balloons via one or more inflation tubes 534.

The balloon inflation system 532 may include a pneumatic cylinder 536 and a piston 538. The pneumatic cylinder 536 may be in fluid communication with the inflation tube 534. Movement of the piston 538 in a first direction within the pneumatic cylinder 536 may cause air or other fluid to pass from the pneumatic cylinder 536, through the inflation tube 534, and into the balloon, thereby inflating the balloon. In contrast, movement of the piston 538 in a second direction within the pneumatic cylinder 536 may create a vacuum within the pneumatic cylinder 536 that causes air or other fluid in the inflation tube 534 to be drawn into the pneumatic cylinder 536, which in turn will draw air or fluid in the balloon to be drawn into the inflation tube 534, thereby deflating the balloon.

In some implementations, the balloon inflation system 532 may be configured as a manually activated system. In such a system, a user would manually activate the system (via interaction with a user input) to either inflate or deflate the balloon. In some implementations, the user input used to activate or deactivate the balloon inflation system 532 may be the input device 530. In such implementations, interaction with the input device 530 may simultaneously control the activation/deactivation of the aspiration and the balloon inflation system 532.

In other implementations, the balloon inflation system 532 may be an automatic system that is automatically activated or deactivated in response to the activation or deactivation of another component of the system. Configuring the balloon inflation system 532 as an automatic system may help ensure that the distal end of the aspiration catheter is centered within a vessel lumen or otherwise spaced apart from a vessel wall prior to starting aspiration. Such an automatic or automated balloon inflation system may mitigate the risk of a user forgetting to inflate the balloon prior to aspiration.

In some implementations, the control 520 and/or the SDU 212 may include sensors or control circuitry that detects user interaction with the input device 530 to activate the valve 526. Prior to opening the valve 526, the control 520 and/or the SDU 212 may activate the balloon inflation system 532 so as to move the piston 538 within the pneumatic cylinder 536 to inflate the balloon. In some implementations, the control 520 and/or the SDU 212 may be able to confirm proper inflation pressure within the balloon and/or balloon integrity (no leaks) through the use of pressure and/or flow sensors in an inflation circuit. Once the balloon is inflated (and optional confirmation thereof is obtained), the control 520 and/or the SDU 212 may activate the valve 526 to initiate aspiration. The control 520 and/or the SDU 212 may also include a bypass system that allows a user to disable or bypass the automatic balloon inflation system if desired for a particular application.

As an alternative to the implementation of FIG. 13, any of the balloons disclosed herein may be inflated and/or deflated with other suitable means, device, or systems. For instance, a syringe (with a barrel and a plunger) may be used. The barrel may be in fluid communication with the balloon. Movement of the plunger within the barrel may cause air, CO2, or other fluid to be inserted into or withdrawn from within the balloon, thereby inflating or deflating the balloon.

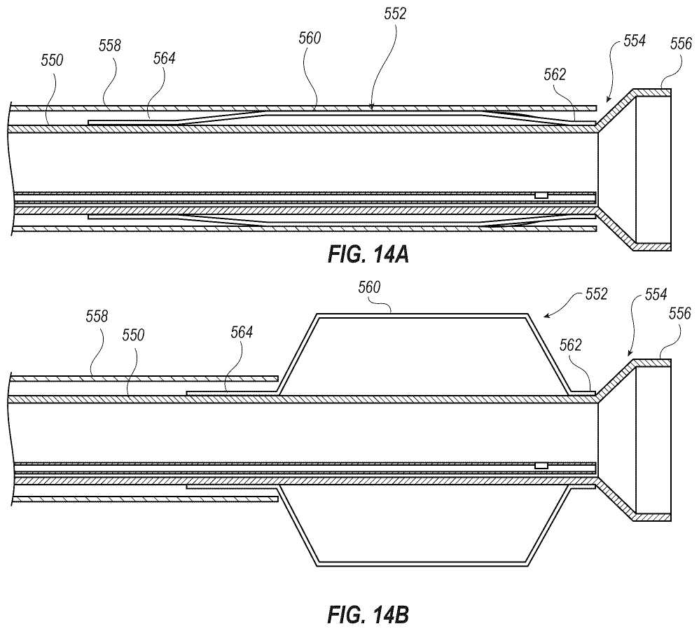

FIGS. 14A-14B illustrates another example implementation of an aspiration catheter 550 with a centering assembly 552 mounted adjacent to a distal end 554 of the aspiration catheter 550. The aspiration catheter 550 may be the same as or similar to the other aspiration catheters disclosed herein, and particularly the aspiration catheters 440, 470, in that the aspiration catheter 550 includes a distal tip 556 that is wider than the rest of the aspiration catheter 550. For instance, the tip 556 may have an outer diameter that is about 2 French, 4 French, 6 French, other dimension larger than the diameter of the rest of the aspiration catheter 550.

The larger dimension of the distal tip 556 may be approximately equal to the outer dimension of a sheath 558 that is disposed around a portion of the length of the aspiration catheter 550 that has an outer diameter that is smaller than the distal tip 556. As a result, the distal tip 556 and the outer sheath 558 may present a generally uniform outer dimension, as shown in FIG. 14A.

The centering assembly 552 of FIGS. 14A and 14B employs a shape memory material, such as Nitinol, that can be moved between a retracted configuration and a deployed configuration. In the illustrated implementation, the centering assembly 552 includes a plurality of legs 560 disposed around the outer circumference of the aspiration catheter 550. Each of the legs 560 includes a distal end 562 that is connected to the outer surface of the aspiration catheter 550 at or adjacent to the distal end 554 thereof. Each of the legs 560 also includes a proximal end 564 that is positioned on or adjacent to the outer surface of the aspiration catheter 550, but is not attached thereto, thereby allowing the proximal ends 564 to move relative to the aspiration catheter 550.

In some cases, the legs 560 are separate and distinct from one another. In other cases, the legs are attached to one another or are portions of an integrated component. For instance, the distal ends 562 of the legs 560 may be attached to one another with a ring that extends around the outer surface of the aspiration catheter 550. Similarly, the proximal ends of the legs 560 may be attached to one another with a ring that extends around the outer surface of the aspiration catheter 550. In other embodiments, the centering assembly 552 may be formed from a tube that has cuts formed therein to define the legs 560.

The shape memory material used to form the centering assembly 550/legs 560 may have a memory shape as shown in FIG. 14B. The illustrated memory shape is merely exemplary and other memory shapes are contemplated. The memory shape may correspond to the deployed configuration of the centering assembly 550. The shape memory material may also be pliable or flexible enough to enable the centering assembly 550/legs 560 to be collapsed into the retracted configuration as shown in FIG. 14A. As can be seen from a comparison between FIGS. 14A and 14B, movement of the centering assembly 550/legs 560 between the retracted and deployed configurations changes the outer diameter of the centering assembly 550/legs 560 and the position of the proximal ends 564 along the length of the aspiration catheter 550.

In the retracted configuration, the outer diameter of the centering assembly 550/legs 560 is smaller (e.g., smaller than the outer diameter of the distal tip 556) and the proximal ends 564 are positioned more proximal along the length of the aspiration catheter 550 (e.g., further from the distal tip 556). In contrast, in the deployed configuration, the proximal ends 564 are positioned more distally along the length of the aspiration catheter 550 (e.g., closer to the distal tip 556). Due to the fixed position of the distal ends 562 and the movable position of the proximal ends 564 along the length of the aspiration catheter 550 and the memory shape of the centering assembly 550/legs 560, the diameter of the centering assembly 550/legs 560 is larger in the deployed configuration than in the retracted configuration.

The centering assembly 550/legs 560 may be held in the retracted configuration by the sheath 558, as shown in FIG. 14A. For instance, distal ends of the legs 560 may be attached to the aspiration catheter 550 and the legs 560 may be compressed towards the aspiration catheter 550 to the position shown in FIG. 14A. The sheath 558 may be slid over the compressed legs 560 to prevent the legs 560 from returning to the memory shape. Once the distal end 554 of the aspiration catheter 550 is positioned in a desired location within a vessel, the sheath 558 may be partially retracted (e.g., to the position shown in FIG. 14B) to at least partially uncover the legs 560. When the sheath 558 is removed from over the legs 560 and no longer retrains the legs 560 in the retracted configuration, the legs 560 are able to automatically return to the memory shape shown in FIG. 14B. That is, the diameter of the legs 560 will expand and the proximal ends 564 will move distally.

In the deployed configuration, the legs 560 may engage the vessel wall and center the aspiration catheter 550 within the vessel lumen, thereby limiting or preventing the vessel wall from being drawn into the aspiration catheter 550 during aspiration.

Once the aspiration procedure is completed, the legs 560 may be returned to the retracted configuration to allow for removal of the aspiration catheter 550 from the vessel. In some implementations, the legs 560 may be shaped so that distal movement of the sheath 558 allows the sheath 558 to slide over the legs 560 and compress the legs back to the retracted configuration. In other implementations, the proximal ends 564 may be connected to a retraction device, such as a cable. Proximal movement of the retraction device may move the proximal ends 564 proximally along the length of the aspiration catheter 550, which can cause the legs 560 to drawn back to the retracted configuration. Once the legs 560 are in the retracted configuration, the sheath 558 may be moved distally over the retracted legs 560 to hold them in the retracted configuration while the aspiration catheter 550 is removed from the vessel.

While the centering assembly 552 has been described as having a plurality of legs 560, it will be appreciated that this is merely exemplary. In other implementations, different types of expandable features may be used in place of or in addition to the legs 560. For instance, the centering assembly 552 may include a self-expandable stent, braided stents, or braids that expand when unsheathed.

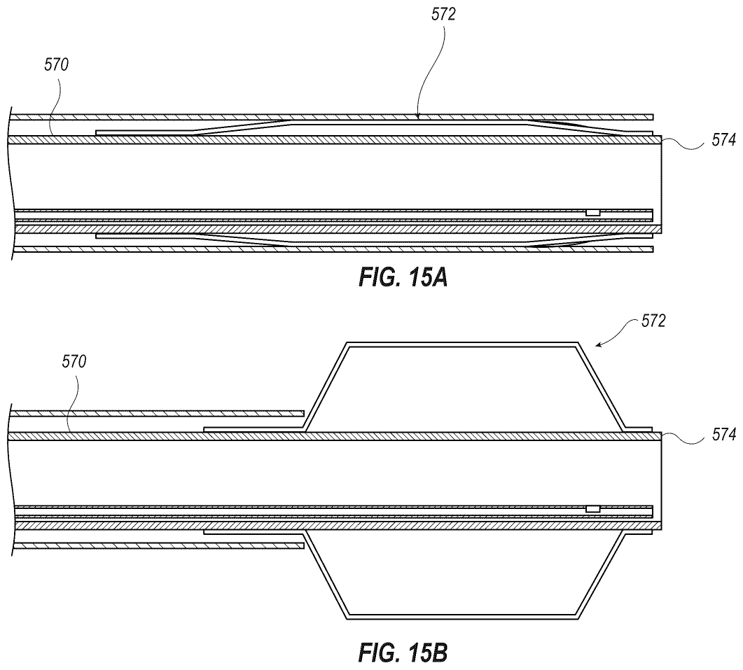

FIGS. 15A-15B illustrate another example implementation of an aspiration catheter 570 with a centering assembly 572. The aspiration catheter 570 and the centering assembly 572 are the same as the aspiration catheter 550 and the centering assembly 552 with one exception. Specifically, in contrast to the distal tip 556 of the aspiration catheter 550 that is wider than the rest of the aspiration catheter 550, the distal end/tip 574 of the aspiration catheter 550 has the same width as the rest of the aspiration catheter 570.

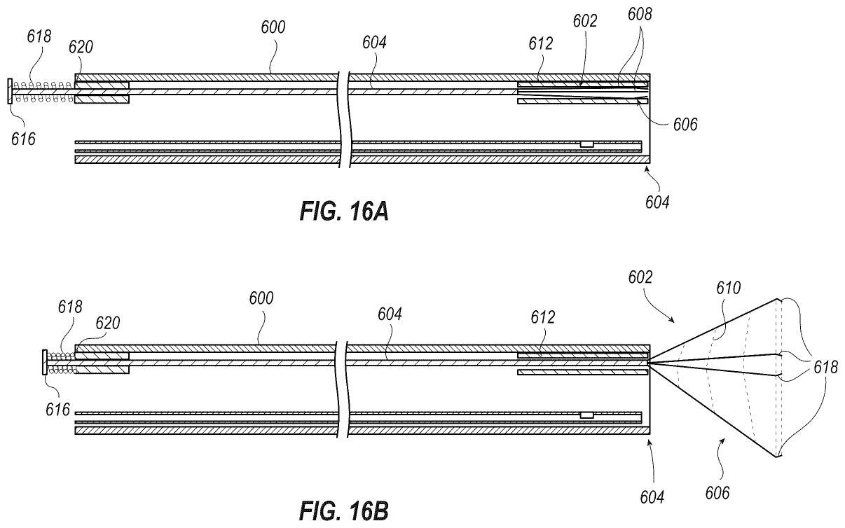

FIGS. 16A-16B illustrate another example implementation of an aspiration catheter 600 with a centering assembly 602 that can be at least partially deployed from a distal end 604 of the aspiration catheter 600. The aspiration catheter 600 may be the same as or similar to the other aspiration catheters disclosed herein.

The centering assembly 602 of FIGS. 16A and 16B includes a skirt 606 that can be selectively moved between a retracted configuration as shown in FIG. 16A and a deployed configuration as shown in FIG. 16B. The skirt 606 may be formed of a plurality of legs 608 that may be formed of flexible wires formed of metals, metal alloys (e.g., Nitinol), plastics, or the like. In some implementations, as illustrated in FIG. 16B, the skirt 606 may also include a material 610 (e.g., silicone, latex, etc.) that extends over, around, and/or between the legs 608. The material 610 may be flexible enough to be movable between the retracted and deployed configurations. In some implementations, the material 610 is configured to form a canopy and the legs 608 function as a supporting structure to the canopy.

The centering assembly 602 may include sleeve 612. At least a portion of the sleeve 612 may be disposed adjacent to the distal end of the catheter 600. The skirt 606 may be partially or fully contained within the sleeve 612 when the skirt 606 is in the retracted configuration, as shown in FIG. 16B.

A proximal end of the skirt 606 (or legs 608 thereof) may be connected to an actuator shaft 614. The actuator shaft 614 may extend proximally through at least a portion of the length of the catheter 600. The actuator shaft 614 may include a shoulder 616. A biasing member 618 (e.g., a coil spring) may be disposed between the shoulder 616 and a fixed reference surface 620. In some implementations, the fixed reference surface 620 is a proximal end of the sleeve 620. In other implementations, the fixed reference surface 620 may be another surface that is located at a fixed position (e.g., along the length of the catheter 600).

The biasing member 618 may apply forces against the fixed references surface 620 and the shoulder 616. Because the fixed reference surface 620 is fixed in place, the forces from the biasing member 618 will bias or tend to move the actuator shaft 614 in a proximal direction. This will cause the skirt 606 to be drawn into or remain in the retracted configuration. On the other hand, when a distal force is applied to the shoulder 616 sufficient to overcome the force of the biasing member 618, the actuator shaft 614 will moved distally relative to the catheter 600.

Distal movement of the actuator shaft 614 will cause the skirt 606 to advance distally out of the distal end 604 of the catheter 600 (and the optional sleeve 612). As the skirt 606 moves out of the catheter 600/sleeve 612, the legs 608 thereof may expand into the deployed configuration shown in FIG. 16B. For instance, the legs 608 may be formed of a shape memory material (e.g., Nitinol) that may have a memory shape as shown in FIG. 16B. The illustrated memory shape is merely exemplary and other memory shapes are contemplated.

The shape memory material used to form the legs 608 may also be pliable or flexible enough to enable the legs 560 to be collapsed into the retracted configuration as shown in FIG. 16A. As can be seen from a comparison between FIGS. 16A and 16B, movement of the legs 560 between the retracted and deployed configurations changes the outer diameter of the centering assembly 602.

In the deployed configuration, the outer diameter of the centering assembly 602 may be large enough to cause the skirt 606 to engage the vessel wall. When the skirt 606 engage the vessel wall, the centering assembly 602 may position the distal end 604 of the catheter 600 near the center of the vessel or at least space the distal end 604 of the catheter 600 away from the vessel wall. To facilitate this centering, at least some of the legs 608 may have different lengths from one another. For instance, as shown in FIG. 16B, if the sleeve 612, the actuator shaft 614, and/or the skirt 606 are off-center from the catheter 600, some of the legs 608 may be longer than others so that the centering assembly 602 may still center the distal end 604 of the catheter 602 in the vessel.

Once the aspiration procedure is completed, the skirt 606 may be returned to the retracted configuration to allow for removal of the aspiration catheter 600 from the vessel. For instance, the pressure applied to the shoulder 616 may be removed, thereby allowing the biasing member 618 to expand. The expansion of the biasing member 618 will move the actuating shaft 614 proximally, which in turn will cause the skirt 606 to be retracted back into the distal end 604 of the catheter 600 (and the optional sleeve 612). Once the skirt 606 is in the retracted configuration, the catheter 600 can be removed from the vessel.

Although the systems for aspirating thrombus described herein are predominantly focused on aspiration, the systems may also, or alternatively, be configured for injecting or infusing fluids, with or without drugs, and may incorporate related features described in U.S. Pat. No. 10,716,583, issued Jul. 21, 2020, and entitled, “Systems and Methods for Removal of Blood and Thrombotic Material” and U.S. Pat. No. 10,492,805, issued Dec. 3, 2019, and entitled, “Systems and Methods for Thrombosis and Delivery of an Agent.”

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, clement, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

Following are some further example embodiments of the invention. These are presented only by way of example and are not intended to limit the scope of the invention in any way. Further, any example embodiment can be combined with one or more of the example embodiments.

Embodiment 1. An aspiration system includes an elongate shaft, a supply lumen, an aspiration lumen, an orifice, and a centering assembly. The elongate shaft is configured for placement within a blood vessel and has a distal end and an outer surface. The supply lumen and the aspiration lumen each extend along the shaft. The supply lumen has a proximal end and a distal end and the aspiration lumen has a proximal end and a distal opening. The orifice is at or near the distal end of the supply lumen and is configured to allow injection of pressurized fluid into the aspiration lumen at or near the distal end of the aspiration lumen when the pressurized fluid is delivered through the supply lumen. The centering assembly is configured for centering the distal end of the elongate shaft within the blood vessel to prevent the blood vessel from being drawn into the aspiration lumen and coming into close proximity to the orifice of the supply lumen during aspiration.

Embodiment 2. The aspiration system of embodiment 1, wherein the centering assembly comprises a balloon disposed adjacent to the distal end of the elongate shaft, the balloon being selectively inflatable and deflatable.

Embodiment 3. The aspiration system of embodiment 2, wherein the balloon has a proximal end and a distal end, the distal end of the balloon being attached to the outer surface of the elongate shaft.

Embodiment 4. The aspiration system of embodiment 3, wherein the distal end of the balloon is folded under and attached to the outer surface of the elongate shaft so as to form a rounded distal region of the balloon when the balloon is inflated.

Embodiment 5. The aspiration system of embodiment 3, wherein the proximal end of the balloon tapers proximally towards the outer surface of the elongate shaft.

Embodiment 6. The aspiration system of embodiment 3, wherein the proximal end of the balloon is attached to the outer surface of the elongate shaft.

Embodiment 7. The aspiration system of embodiment 2, further comprising a coaxial shaft disposed around at least a portion of a length of the elongate shaft.

Embodiment 8. The aspiration system of embodiment 7, wherein the proximal end of the balloon is attached to the coaxial shaft.

Embodiment 9. The aspiration system of embodiment 2, wherein the elongate shaft comprises a distal tip at the distal end thereof, the distal tip having an outer diameter that is larger than the outer diameter of another portion of the elongate shaft.

Embodiment 10. The aspiration system of embodiment 9, wherein the balloon in a deflated configuration has an outer dimension that is approximately the same as or smaller than the outer diameter of the distal tip.

Embodiment 11. The aspiration system of embodiment 2, wherein the balloon, when inflated, has a distal end that is disposed proximal to, aligned with, or distal to the distal end of the elongate shaft.

Embodiment 12. The aspiration system of embodiment 2, wherein the balloon extends around an entire outer circumference of the elongate shaft.

Embodiment 13. The aspiration system of embodiment 2, wherein the balloon comprises a plurality of balloons.

Embodiment 14. The aspiration system of embodiment 13, wherein the plurality of balloons are spaced apart from one another about an outer circumference of the elongate shaft.

Embodiment 15. The aspiration system of embodiment 2, further comprising a balloon inflation system for inflating the balloon.

Embodiment 16. The aspiration system of embodiment 15, wherein the balloon inflation system comprises a piston and a pneumatic cylinder in fluid communication with the balloon.

Embodiment 17. The aspiration system of embodiment 15, wherein the balloon inflation system is manually operated to inflate or deflate the balloon.

Embodiment 18. The aspiration system of embodiment 15, wherein the balloon inflation system is automatically operated in response to activation of another component of the aspiration system.

Embodiment 19. The aspiration system of embodiment 1, wherein the centering assembly comprises a plurality of legs disposed adjacent to the distal end of the elongate shaft, the plurality of legs being movable between a retracted configuration and a deployed configuration.

Embodiment 20. The aspiration system of embodiment 19, wherein the plurality of legs are formed of a shape memory material and have a memory shape, the memory shape corresponding to the deployed configuration.

Embodiment 21. The aspiration system of embodiment 19, wherein the plurality of legs have proximal and distal ends.

Embodiment 22. The aspiration system of embodiment 21, wherein the distal ends of the plurality of legs are attached to the outer surface of the cognate shaft adjacent to the distal end of the elongate shaft.

Embodiment 23. The aspiration system of embodiment 22, wherein the proximal ends of the plurality of legs are disposed adjacent to the outer surface of the elongate shaft and are configured for movement along a portion of a length of the elongate shaft.

Embodiment 24. The aspiration system of embodiment 19, wherein the centering assembly is formed of a tube with cuts therein to define the plurality of legs.

Embodiment 25. An aspiration system includes an elongate shaft, a supply lumen, an aspiration lumen, an orifice, and a centering assembly. The elongate shaft is configured for placement within a blood vessel and has a distal end with an opening at the distal end. The supply lumen and the aspiration lumen each extend along the shaft. The supply lumen has a proximal end and a distal end and the aspiration lumen has a proximal end and a distal opening. The orifice is at or near the distal end of the supply lumen and is configured to allow injection of pressurized fluid into the aspiration lumen at or near the distal end of the aspiration lumen when the pressurized fluid is delivered through the supply lumen. The centering assembly is disposed at the distal end of the elongate shaft and comprises one or more balloons that are selectively inflatable to center the distal end of the elongate shaft within the blood vessel to prevent the blood vessel from being drawn into the aspiration lumen and into close proximity to the orifice of the supply lumen during aspiration.

Embodiment 26. The aspiration system of embodiment 25, wherein each of the one or more balloons has a proximal end and a distal end, the distal end thereof being attached to the outer surface of the cognate shaft.

Embodiment 27. The aspiration system of embodiment 26, wherein the distal ends of the one or more balloons is folded under and attached to the outer surface of the elongate shaft so as to form a rounded distal region of the one or more balloons when the one or more balloons are inflated.

Embodiment 28. The aspiration system of embodiment 26, wherein the proximal ends of the one or more balloons tapers proximally towards the outer surface of the cognate shaft.

Embodiment 29. The aspiration system of embodiment 26, wherein the proximal ends of the one or more balloons are attached to the outer surface of the elongate shaft.

Embodiment 30. The aspiration system of embodiment 25, further comprising a coaxial shaft disposed around at least a portion of a length of the elongate shaft.

Embodiment 31. The aspiration system of embodiment 30, wherein the proximal ends of the one or more balloons are attached to the coaxial shaft.

Embodiment 32. The aspiration system of embodiment 25, wherein the cognate shaft comprises a distal tip at the distal end thereof, the distal tip having an outer diameter of that is larger than the outer diameter of another portion of the elongate shaft.

Embodiment 33. The aspiration system of embodiment 32, wherein the one or more balloons in a deflated configuration have an outer dimension that is approximately the same as or smaller than the outer diameter of the distal tip.

Embodiment 34. The aspiration system of embodiment 25, wherein the one or more balloons, when inflated, have a distal end that is disposed proximal to, aligned with, or distal to the distal end of the elongate shaft.

Embodiment 35. The aspiration system of embodiment 25, wherein the one or more balloons extend around an entire outer circumference of the elongate shaft.

Embodiment 36. The aspiration system of embodiment 25, wherein the one or more balloons comprise a plurality of balloons.

Embodiment 37. The aspiration system of embodiment 36, wherein the plurality of balloons are spaced apart from one another about an outer circumference of the elongate shaft.

Embodiment 38. The aspiration system of embodiment 25, further comprising a balloon inflation system for inflating the one or more balloons.

Embodiment 39. The aspiration system of embodiment 38, wherein the balloon inflation system comprises a piston and a pneumatic cylinder in fluid communication with the one or more balloons.

Embodiment 40. The aspiration system of embodiment 38, wherein the balloon inflation system is manually operated to inflate or deflate the one or more balloons.

Embodiment 41. The aspiration system of embodiment 38, wherein the balloon inflation system is automatically operated in response to activation of another component of the aspiration system.

Embodiment 42. An aspiration system includes an elongate shaft, a supply lumen, an aspiration lumen, an orifice, and a centering assembly. The elongate shaft is configured for placement within a blood vessel and has a distal end with an opening at the distal end. The supply lumen and the aspiration lumen each extend along the shaft. The supply lumen has a proximal end and a distal end and the aspiration lumen has a proximal end and a distal opening. The orifice is near the distal end of the supply lumen and is configured to allow injection of pressurized fluid into the aspiration lumen at or near the distal end of the aspiration lumen when the pressurized fluid is delivered through the supply lumen. The centering assembly is disposed at the distal end of the elongate shaft and includes a plurality of legs disposed adjacent to the distal end of the elongate shaft. The plurality of legs are movable between a retracted configuration and a deployed configuration to center the distal end of the elongate shaft within the blood vessel to prevent the blood vessel from being drawn into the aspiration lumen and into close proximity to the orifice of the supply lumen during aspiration.

Embodiment 43. The aspiration system of embodiment 42, wherein the plurality of legs are formed of a shape memory material and have a memory shape, the memory shape corresponding to the deployed configuration.

Embodiment 44. The aspiration system of embodiment 42, wherein the plurality of legs have proximal and distal ends.

Embodiment 45. The aspiration system of embodiment 44, wherein the distal ends of the plurality of legs are attached to the outer surface of the elongate shaft adjacent to the distal end of the elongate shaft.

Embodiment 46. The aspiration system of embodiment 45, wherein the proximal ends of the plurality of legs are disposed adjacent to the outer surface of the elongate shaft and are configured for movement along a portion of a length of the elongate shaft.

Embodiment 47. The aspiration system of embodiment 42, wherein the centering assembly is formed of a tube with cuts therein to define the plurality of legs.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. An aspiration system comprising:
an elongate shaft configured for placement within a blood vessel, the elongate shaft having a distal end and an outer surface;
a supply lumen and an aspiration lumen each extending along the shaft, the supply lumen having a proximal end and a distal end, and the aspiration lumen having a proximal end and a distal opening;
an orifice at or near the distal end of the supply lumen, the orifice being configured to allow injection of pressurized fluid into the aspiration lumen at or near the distal end of the aspiration lumen when the pressurized fluid is delivered through the supply lumen; and
a centering assembly for centering the distal end of the elongate shaft within the blood vessel to prevent the blood vessel from being drawn into the aspiration lumen and coming into close proximity to the orifice of the supply lumen during aspiration.

2. The aspiration system of claim 1, wherein the centering assembly comprises a balloon disposed adjacent to the distal end of the elongate shaft, the balloon being selectively inflatable and deflatable.

3. The aspiration system of claim 2, wherein the balloon has a proximal end and a distal end, the distal end of the balloon being attached to the outer surface of the elongate shaft, wherein:
the distal end of the balloon is folded under and attached to the outer surface of the elongate shaft so as to form a rounded distal region of the balloon when the balloon is inflated;
the proximal end of the balloon tapers proximally towards the outer surface of the elongate shaft; or
the proximal end of the balloon is attached to the outer surface of the elongate shaft.

4. The aspiration system of claim 2, further comprising a coaxial shaft disposed around at least a portion of a length of the elongate shaft, wherein the proximal end of the balloon is attached to the coaxial shaft.

5. The aspiration system of claim 2, wherein the elongate shaft comprises a distal tip at the distal end thereof, the distal tip having an outer diameter that is larger than the outer diameter of another portion of the elongate shaft, wherein the balloon in a deflated configuration has an outer dimension that is approximately the same as or smaller than the outer diameter of the distal tip.

6. The aspiration system of claim 2, wherein the balloon:
when inflated, has a distal end that is disposed proximal to, aligned with, or distal to the distal end of the elongate shaft;
extends around an entire outer circumference of the elongate shaft; or
comprises a plurality of balloons spaced apart from one another about an outer circumference of the elongate shaft.

7. The aspiration system of claim 2, further comprising a balloon inflation system for inflating the balloon, wherein the balloon inflation system:
comprises a piston and a pneumatic cylinder in fluid communication with the balloon;
is manually operated to inflate or deflate the balloon; or
is automatically operated in response to activation of another component of the aspiration system.

8. The aspiration system of claim 1, wherein the centering assembly comprises a plurality of legs disposed adjacent to the distal end of the elongate shaft, the plurality of legs being movable between a retracted configuration and a deployed configuration, wherein:
the plurality of legs are formed of a shape memory material and have a memory shape, the memory shape corresponding to the deployed configuration;
the plurality of legs have proximal and distal ends, wherein the distal ends of the plurality of legs are attached to the outer surface of the elongate shaft adjacent to the distal end of the elongate shaft, wherein the proximal ends of the plurality of legs are disposed adjacent to the outer surface of the elongate shaft and are configured for movement along a portion of a length of the elongate shaft; or
the centering assembly is formed of a tube with cuts therein to define the plurality of legs.

9. An aspiration system comprising:
an elongate shaft configured for placement within a blood vessel, the elongate shaft having a distal end with an opening at the distal end;
a supply lumen and an aspiration lumen each extending along the shaft, the supply lumen having a proximal end and a distal end, and the aspiration lumen having a proximal end and a distal opening;
an orifice near the distal end of the supply lumen, the orifice being configured to allow injection of pressurized fluid into the aspiration lumen at or near the distal end of the aspiration lumen when the pressurized fluid is delivered through the supply lumen; and
a centering assembly disposed at the distal end of the elongate shaft, the centering assembly comprising one or more balloons that are selectively inflatable to center the distal end of the elongate shaft within the blood vessel to prevent the blood vessel from being drawn into the aspiration lumen and into close proximity to the orifice of the supply lumen during aspiration.

10. The aspiration system of claim 9, wherein each of the one or more balloons has a proximal end and a distal end, the distal end thereof being attached to the outer surface of the elongate shaft; wherein:
the distal ends of the one or more balloons is folded under and attached to the outer surface of the elongate shaft so as to form a rounded distal region of the one or more balloons when the one or more balloons are inflated;
the proximal ends of the one or more balloons tapers proximally towards the outer surface of the elongate shaft;
the proximal ends of the one or more balloons are attached to the outer surface of the elongate shaft.

11. The aspiration system of claim 9, further comprising a coaxial shaft disposed around at least a portion of a length of the elongate shaft, wherein the proximal ends of the one or more balloons are attached to the coaxial shaft.

12. The aspiration system of claim 9, wherein the one or more balloons extend around an entire outer circumference of the elongate shaft.

13. The aspiration system of claim 9, further comprising a balloon inflation system for inflating the one or more balloons.

14. The aspiration system of claim 13, wherein the balloon inflation system is:
manually operated to inflate or deflate the one or more balloons; or
automatically operated in response to activation of another component of the aspiration system.

15. An aspiration system comprising:
an elongate shaft configured for placement within a blood vessel, the elongate shaft having a distal end with an opening at the distal end;
a supply lumen and an aspiration lumen each extending along the shaft, the supply lumen having a proximal end and a distal end, and the aspiration lumen having a proximal end and a distal opening;
an orifice near the distal end of the supply lumen, the orifice being configured to allow injection of pressurized fluid into the aspiration lumen at or near the distal end of the aspiration lumen when the pressurized fluid is delivered through the supply lumen; and
a centering assembly disposed at the distal end of the elongate shaft, the centering assembly comprising a plurality of legs disposed adjacent to the distal end of the elongate shaft, the plurality of legs being movable between a retracted configuration and a deployed configuration to center the distal end of the elongate shaft within the blood vessel to prevent the blood vessel from being drawn into the aspiration lumen and into close proximity to the orifice of the supply lumen during aspiration.

16. The aspiration system of claim 15, wherein the plurality of legs are formed of a shape memory material and have a memory shape, the memory shape corresponding to the deployed configuration.

17. The aspiration system of claim 15, wherein the plurality of legs have proximal and distal ends.

18. The aspiration system of claim 17, wherein the distal ends of the plurality of legs are attached to the outer surface of the elongate shaft adjacent to the distal end of the elongate shaft.

19. The aspiration system of claim 18, wherein the proximal ends of the plurality of legs are disposed adjacent to the outer surface of the elongate shaft and are configured for movement along a portion of a length of the elongate shaft.

20. The aspiration system of claim 15, wherein the centering assembly is formed of a tube with cuts therein to define the plurality of legs.
